(19) United States
(12) Patent Application Publication
(10) Pub. No.: US 2012/0323082 A1
(43) Pub. Date: Dec. 20, 2012

(54) SUPPORT SYSTEM FOR SURGICAL INSTRUMENT

(76) Inventor: KEITH CATHEY, Tulsa, OK (US)

(21) Appl. No.: 13/164,604

(22) Filed: Jun. 20, 2011

(51) Int. Cl. A61B 1/32

(52) U.S. Cl. 600/227

(57) ABSTRACT

A holding device for a surgical instrument may include a central instrument holding plate, patient attachment straps extending from the holding plate and instrument fasteners attached to the holding plate. In an exemplary application of the holding device the central holding plate may support a weighted speculum. Patient attachment straps may extend from the holding plate and may be configured to surround thighs of the patient. The fasteners may be configured to hold the speculum.

### BACKGROUND OF THE INVENTION

The present invention generally relates to a system for holding a surgical instrument in position on a patient during a surgical procedure.

During some surgical procedures it may be necessary to support a device the holds a portion of a patient's anatomy in a particular fixed position during the procedure. For example, in gynecological procedures a weighted speculum may be employed to maintain an opening that allows a surgeon to gain access to various anatomical elements within the patient's body. The weighted speculum may hold vaginal tissue in a separated and open state. Because of its weight, (approximately 5 pounds) gravitational force may cause the speculum to naturally maintain the tissue into the desired open state. The weight of the speculum may also result in a tendency for the speculum to slip out of the patient.

If a weighted speculum were to slip out of a patient, the speculum would, at a minimum, require re-sterilization, or in a worse case, a falling weighted speculum may drop strike a surgeon or other operating room staff member and cause personal injury. Thus, it is normal practice in such gynecological procedures to assign a surgical assistant a task of holding the speculum to assure that it does not slip out of the patient.

As can be seen, there is a need for a system of maintaining an instrument such as a speculum securely in position without a need for assigning a surgical assistant to the task of holding the instrument in position.

### SUMMARY OF THE INVENTION

In one aspect of the present invention, a holding device for a surgical instrument may comprise: a central instrument holding plate; patient attachment straps extending from the holding plate; and instrument fasteners attached to the holding plate.

In another aspect of the present invention, a device for holding a weighted speculum in position on a patient may comprise: a central holding plate for the speculum; patient-attachment straps extending from the holding plate and configured to surround thighs of the patient; and fasteners attached to the holding plate configured to hold the speculum.

In still another aspect of the present invention, a method of performing a gynecological procedure on a patient may comprise the steps of: providing for placement of fasteners for a weighted speculum in a region adjacent a vaginal opening of the patient; and providing for flexible attachment of the weighted speculum to the fasteners.

These and other features, aspects and advantages of the present invention will become better understood with reference to the following drawings, description and claims.

### BRIEF DESCRIPTION OF THE DRAWINGS

FIGS. 1 and 2 are perspective views of a holding device for a surgical instrument in accordance with an embodiment of the invention; and

FIG. 3 is a perspective view of the holding device of FIGS. 1 and 2 in position on a patient.

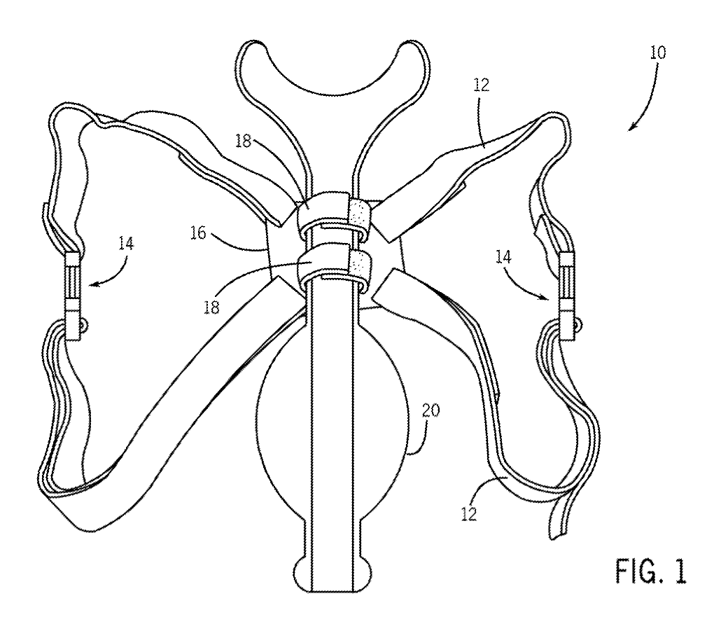

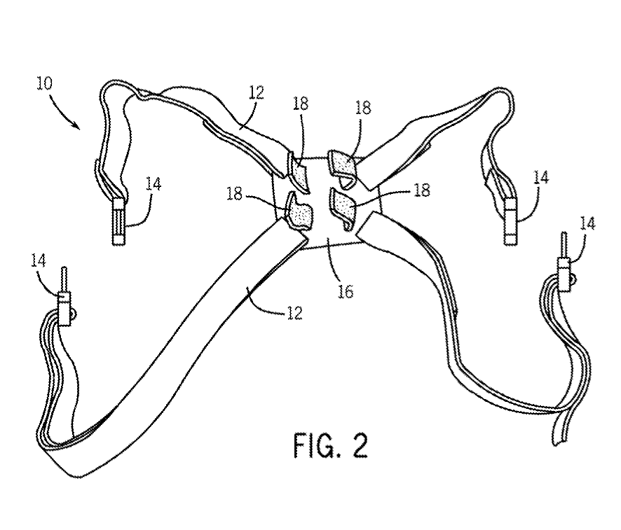

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a holding system in which a holding device may be secured to a patient and an instrument may be attached to the device and held securely in position at a desired location relative to the patient.

Referring now to the Figures, it may be seen that an exemplary embodiment of a holding device 10 may be configured to hold an instrument 20, for example a weighted speculum, in position on a patient. The holding device 10 may comprise patient-attachment straps 12, a central holding plate 16 and instrument fasteners 18.

Advantageously, the holding device 10 may be brought into a surgical setting as a pre-packed sterile unit. The patient-attachment straps 12 may be flexible or elastomeric fabric. The patient-attachment straps 12 may be adjustable in length and may be provided with buckles 14. The central holding plate 16 may be comprised of semi-rigid plastic foam without any sharp edges. All materials used in construction of the holding device 10 should be tolerant of being sterilizing.

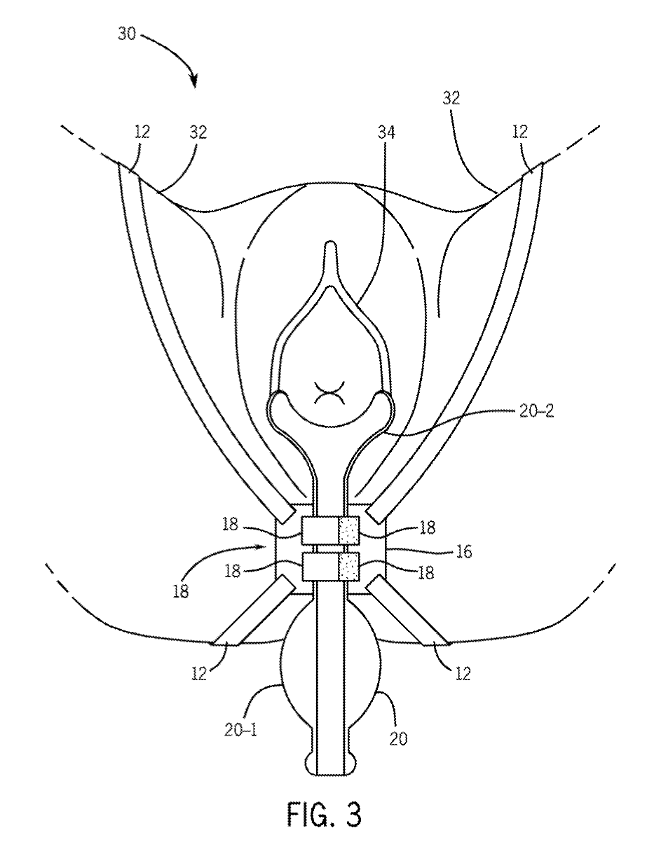

Referring now particularly to FIG. 3, it may be seen that in an exemplary application of the holding device 10, the patient-attachment straps 12 may be placed around thighs 32 of a patient 30. The patient-attachment straps 12 may be adjusted in length so that the central holding plate 16 may be held securely against the patient 30. The surgical instrument 20 may be positioned as desired in the patient 34 and the instrument fasteners 18 may be secured around the instrument 20.

In the exemplary case of the instrument 20 being a weighted speculum, the instrument 20 may be positioned to hold open the patient's vagina. A weighted speculum is constructed with a weighted end 20-1. Gravitational force on the weighted end 20-1 may cause a contact portion 20-2 of the instrument 20 to exert a downward force on the vaginal opening 34 of the patient 30. This downward force may be useful in maintaining an open region through which a surgeon may perform a procedure.

It may be noted that the instrument fasteners 18 may be configured so that they may be engaged with the instrument 20 in a manner that does not preclude the desirable effects of the weighted end 20-1 on the functionality of the instrument 20. In other words, the fasteners 18 may be constructed so that they may be attached to the instrument with enough flexibility as to allow the weighted end 20-1 to continue exerting desired downward force on the contact portion 20-2 as described above.

Advantageously, the instrument fasteners 18 may be constructed so that their engagement force with the instrument 20 may be adjusted as desired to allow for full functionality of the instrument 20. For example, the fasteners may comprise hook and loop material (e.g., Velcro®). Alternatively, the fasteners 18 may comprise adjustable straps (not shown) that fasten together with snaps or buckles (not shown).

It should be understood, of course, that the foregoing relates to exemplary embodiments of the invention and that modifications may be made without departing from the spirit and scope of the invention as set forth in the following claims.

What is claimed is:

1. A holding device for a surgical instrument, comprising:
a central instrument holding plate;
patient attachment straps extending from the holding plate; and
instrument fasteners attached to the holding plate.

2. The holding device of claim 1 wherein the instrument fasteners are configured for flexible engagement with the surgical instrument.

3. The holding device of claim 2 wherein the instrument fasteners are comprised of hook and loop material.

4. The holding device of claim 1 wherein the patient-attachment straps are comprised of flexible fabric.

5. The holding device of claim 1 wherein the patient-attachment straps comprise buckles.

6. A device for holding a weighted speculum in position on a patient comprising:
a central holding plate for the speculum;
patient attachment straps extending from the holding plate and configured to surround thighs of the patient; and
fasteners attached to the holding plate, the fasteners configured to hold the speculum.

7. The device of claim 6 wherein the fasteners are configured for flexible engagement with the speculum.

8. The device of claim 7 wherein the fasteners are comprised of hook and loop material.

9. A method of performing a gynecological procedure on a patient comprising the steps of:
providing for placement of fasteners for a weighted speculum in a region adjacent a vaginal opening of the patient; and
providing for flexible attachment of the weighted speculum to the fasteners.

10. The method of claim 9 wherein the step of providing for placement of fasteners comprises providing for placement of flexible straps around thighs of the patient to hold a central holding plate against the patient in the region adjacent the vaginal opening, wherein the holding plate supports the fasteners.
